(19) United States
(12) Patent Application Publication
(10) Pub. No.: US 2025/0391573 A1
(43) Pub. Date: Dec. 25, 2025

(54) GENERATING A COMPOSITE RESULT USING INFLAMMATION DATA

(71) Applicant: Sequelae, Inc., Minneapolis, MN (US)

(72) Inventors: James LaBelle, Encinitas, CA (US); Douglas A. Granger, Coto de caza, CA (US); Benjamin C. Wiegand, Yardley, PA (US); Steven J. Catani, Athens, GA (US)

(73) Assignee: Sequelae, Inc., Minneapolis, MN (US)

(21) Appl. No.: 19/242,194

(22) Filed: Jun. 18, 2025

Related U.S. Application Data
(60) Provisional application No. 63/662,281, filed on Jun. 20, 2024.

(51) Int. Cl. G16H 50/30; G01N 33/68; G16H 10/60; G16H 80/00

(52) U.S. Cl. CPC G16H 50/30; G01N 33/6893; G16H 10/60; G16H 80/00; G01N 2333/4737; G01N 2800/7095

(57) ABSTRACT

Methods and systems for managing an adverse health condition are provided. A digital communication network (DCN) is provided for a plurality of members of a population to interact with each other. A C-reactive protein (CRP) sample and at least one other inflammatory biomarker sample are received from an individual member. The CRP sample and the other inflammatory biomarker sample are analyzed and a CRP level and an inflammatory biomarker level are determined from the analysis. A composite result based on the CRP level and the inflammatory biomarker level is generated.

### CROSS REFERENCE TO RELATED APPLICATIONS

This application claims the benefit of U.S. Provisional Application No. 63/662,281, filed on Jun. 20, 2024, which application is incorporated herein by reference in its entirety.

### BACKGROUND

Various embodiments relate generally to health care systems, methods, devices and computer programs and, more specifically, relate to generating a composite result using inflammation data.

This section is intended to provide a background or context. The description may include concepts that may be pursued, but have not necessarily been previously conceived or pursued. Unless indicated otherwise, what is described in this section is not deemed prior art to the description and claims and is not admitted to be prior art by inclusion in this section.

C-reactive protein (CRP) is useful in measuring system inflammation in an individual member. CRP may correlate with other inflammatory markers and can be a good predictor of the progression of many diseases. However, there are instances when CRP and other inflammatory markers may not correlate. As these instances appear relative rare, many medical testing systems rely on CRP alone.

What is needed is a way to control progression of a health condition using improved and combined inflammation data.

### SUMMARY

Example aspects of the present disclosure include:
A method for managing an adverse health condition according to at least one embodiment of the present disclosure comprises providing a digital communication network (DCN) for a plurality of members of a population to interact with each other and the DCN; receiving a C-reactive protein (CRP) sample from an individual member of a plurality of members of a population; receiving at least one other inflammatory biomarker sample from the individual member; analyzing the CRP sample and the at least one other inflammatory biomarker; determining a CRP level from the CRP sample and an inflammatory biomarker level from the at least one other inflammatory biomarker sample; generating a composite result based on the CRP level and the inflammatory biomarker level; and providing the composite result to the individual member via the DCN, wherein the composite result correlates to whether the individual member is experiencing inflammation.

Any of the aspects herein, wherein the composite result comprises at least one of: an index created by adding values of the CRP level and the inflammatory biomarker level, an index created by providing a highest level of an index created by adding values of the CRP level and the inflammatory biomarker level, a spider graph plotting the levels of an index created by adding values of the CRP level and the inflammatory biomarker level, an index calculated from the area inside the spider graph plotting the levels of an index created by adding values of the CRP level and the inflammatory biomarker level, and an index calculated as the average plus x times the standard deviation of the levels of an index created by adding values of the CRP level and the inflammatory biomarker level.

Any of the aspects herein, wherein the CRP level and the inflammatory biomarkers level are normalized.

Any of the aspects herein, further comprising normalizing the values of the CRP level and the inflammatory biomarker level.

Any of the aspects herein, further comprising comparing an index of the composite result with a predetermined threshold and determining that the individual member is experiencing inflammation when the index meets the predetermined threshold.

Any of the aspects herein, wherein the at least one other inflammatory biomarker is at least one of: TNF-alpha, IL-1 Beta, IL-6, IL-8, IL-10, IL-17, IL-17a, IL-12, INF, IL-23, Erythrocyte Sedimentation Rate, Plasma Viscosity, Fibringen, Ferritin, Procalcitonin, Psychometric Instruments of depression, anxiety, stress, and cognitive load, and Fasting and postprandial Insulin.

Any of the aspects herein, wherein the adverse health condition comprises at least one of chronic systemic inflammation, malaise, low energy, a disease, a health risk, social dysfunction, or a prodromal disease.

Any of the aspects herein, wherein the disease comprises at least one of obesity, diabetes, rheumatoid arthritis, Crohn's disease, psoriasis, eczema, cardiovascular disease, congestive heart failure, chronic obstructive pulmonary disease, asthma, depression, or anxiety.

Any of the aspects herein, wherein the health risk is at least one of falling or becoming infected with an illness.

Any of the aspects herein, wherein the method is used to measure chronic systemic inflammation.

Any of the aspects herein, wherein the method is used to manage the adverse health condition in the population.

Any of the aspects herein, wherein the method is used to reduce the health risk in the population.

Any of the aspects herein, further comprising sending a question to the individual member from the DCN, wherein the question is designed to induce a reflection based on the composite result.

Any of the aspects herein, wherein the question is further designed to induce the reflection based on lifestyle and environmental factors that caused the composite result.

Any of the aspects herein, further comprising reporting the composite result in the DCN.

Any of the aspects herein, further comprising enabling the individual member to share the composite result with other members in the DCN.

Any of the aspects herein, further comprising allowing the other members in the DCN to comment upon the composite result.

Any of the aspects herein, wherein enabling the individual member to share the composite result comprises at least one of: sharing the results via a conversation bot; allowing the member to discuss the results with the conversation bot; and drafting, by the conversation bot, a communication to share with the other members in the DCN.

Any of the aspects herein, wherein the conversation bot is designed to have a conversation configured to induce a reflection about the composite result in the member.

A system according to at least one embodiment of the present disclosure comprises a computer processor; a data repository in communication with the computer processor and storing: a C-reactive protein (CRP) sample, a CRP level, an inflammatory biomarker sample, an inflammatory biomarker level, and a composite result, a composite result generator configured to receive the CRP level and the inflammatory biomarker level as input and to output the composite result; a digital communications network which, when executed by the computer processor, provides a network for members of a population to interact with each other; and a server controller which, when executed by the computer processor: provide a digital communication network (DCN) for a plurality of members of a population to interact with each other and the DCN; receive the CRP sample from an individual member of a plurality of members of a population; receive the inflammatory biomarker sample from the individual member; analyze the CRP sample and the at least one other inflammatory biomarker; determine the CRP level from the CRP sample and the inflammatory biomarker level from the inflammatory biomarker sample; generate the composite result based on the CRP level and the inflammatory biomarker level; and provide the composite result to the individual member via the DCN.

### BRIEF DESCRIPTION OF THE DRAWINGS

Aspects of the described embodiments are more evident in the following description, when read in conjunction with the attached Figures.

FIG. 1 shows a simplified block diagram of devices, in accordance with one or more embodiments.

FIG. 2 is a logic flow diagram that illustrates the operation of a method, in accordance with one or more embodiments.

FIG. 3 is a logic flow diagram that illustrates the operation of a method, in accordance with one or more embodiments.

FIG. 4A shows an example of a computing system, in accordance with one or more embodiments.

FIG. 4B shows an example of a network, in accordance with one or more embodiments.

### DETAILED DESCRIPTION

Systems and methods are provided in which CRP levels and other or additional inflammatory biomarker levels are measured to determine if an individual member is experiencing inflammation. The inflammatory biomarker levels may be useful in instances where the CRP levels do not correlate with the inflammatory biomarker levels. In other words, the inflammatory biomarker levels can indicate inflammation when the CRP does not in some instances. By looking at the combined results of the CRP testing and testing for other inflammatory biomarkers, the inflammation data provides a fuller perspective of the individual member's health conditions.

Additionally, the individual member's combined results of CRP testing and testing for other inflammatory biomarker can be used to provide the individual member's experience as a social aspect within a community such as a digital communication network (DCN). As a social process, value can be placed on those members of a community that have relevant experience to inflammation. Reflections from the individual members on, for example, the results of their CRP and/or inflammatory biomarker testing can be induced in order to help elevate the aspects of the experience that may be more instructional to the community as a whole. This can help increase the number of members of the community thinking about an action, for example, creating an atmosphere where people can learn about others in who have undergone a change to, for example, improve their inflammation that they are considering.

The community (through, for example, the DCN) can also be used to encourage lifestyle interventions, which are inherently safe. Such community may use the philosophy that any action now is preferred to a “better” action later and also support the concept that ideas and communication are healthcare. Conventionally, lifestyle change has been looked at as in individual pursuit, such as plans personalized just for the individual. Further, medicine is typically a one-on-one activity (reinforced by the privacy concepts the system is based on). In the present disclosure, lifestyle change is seen as highly driven by social parameters and the impact on social parameters is critical.

Conventional lifestyle applications may tell individuals the “right thing to do”, which could be right, but given the complexity of the lifestyle change, is likely not to occur. Often, if the lifestyle change suggested works, they can make the individual more dependent on things outside the individual's control. On the other hand, communities share experiences, not expertise, which individuals can try and if they work for them, is a success. In some cases, success can range from slowing the progression of adverse conditions to managing a disease, or the overall risk level in a population.

Lifestyle change may be supported by communities that provide support, ideas, and, in the case of these ideas, access to tools to provide objective data to make meaningful lifestyle changes in an individual member or members of a population. For example, in a community where a person is a peer, the actions they take and learn from are that their volition and may result in increased agency (or autonomy) or self-efficacy. This not only increase the chances of continuous lifestyle improvement, but improved outcomes throughout the heath system.

Further, online communities such as the DCN can be provided so that people can learn about healthy lifestyle practices and work to improve their health between clinical touchpoints, such as office visits. To help incentivize healthy behaviors, individuals can use tools like in-home tests and biosensors that measure how well their health actions are working. For example, individual members can use CRP testing and/or inflammatory biomarker testing.

Thus, it is desirable to provide systems and methods that can be used to generate a composite result for an individual member based on their CRP level and inflammatory biomarker level and using the composite result to determine if the individual member is experiencing inflammation. If the individual member is experiencing inflammation, then an action such as sending a question to the individual member to induce reflection in the individual member, determining and sending an intervention or recommendation to the individual member, enabling the individual member to post their composite result to the DCN, etc. may be provided.

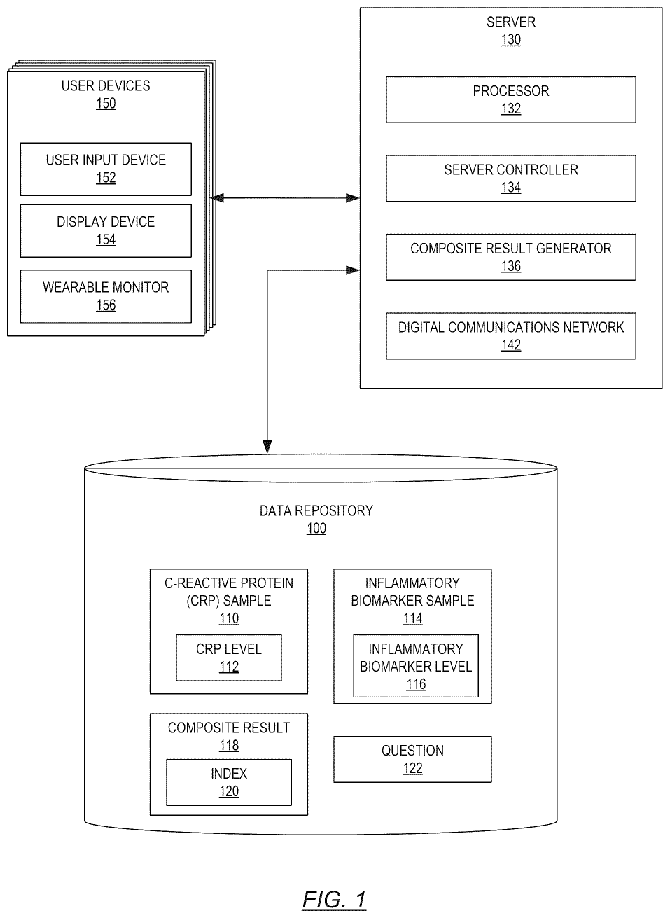

The system shown in FIG. 1 includes a data repository (100). The data repository (100) is a type of storage unit or device (e.g., a file system, database, data structure, or any other storage mechanism) for storing data (described below). The data repository (100) may include multiple different, potentially heterogeneous, storage units and/or devices.

The data repository (100) stores C-reactive protein (CRP) samples (110). The CRP is a protein produced by the liver and can be used to measure inflammation in an individual member of the population. The CRP samples (110) can be used to measure a CRP level (112). The CRP level (112) increases when inflammation is present or increases in the individual member.

The data repository (100) also stores inflammatory biomarker samples (114). The inflammatory biomarker samples (114) may be, for example, any one of the following or a combination of: TNF-alpha, IL-1 Beta, IL-6, IL-8, IL-10, IL-12, IL-17, IL-17a, INF, IL-23, Erythrocyte Sedimentation Rate, Plasma Viscosity, Fibringen, Ferritin, Procalcitonin, Psychometric Instruments of depression, anxiety, stress, cytokines, antibodies, heart rate variability, and cognitive load, and fasting and postprandial insulin. The inflammation biomarker samples (114) can also be used to measure an inflammation biomarker level (116).

The CRP samples (110) and/or the inflammatory biomarker samples (114) can be obtained in a variety of ways. For example, the CRP samples (110) and/or the inflammatory biomarker samples (114) can be measured and evaluated using a blood, urine, stool, saliva, breath, or soft tissue sample from a user or member of a population or from heart-rate meters, accelerometer samples, body temperature as well as other signals derived from wearables. The CRP samples (110) and/or the inflammatory biomarker samples (114) can also be derived from psychometric instruments and EMA-derived data. The CRP samples (110) and/or the inflammatory biomarker samples (114) can be obtained from testing at home or at a clinic.

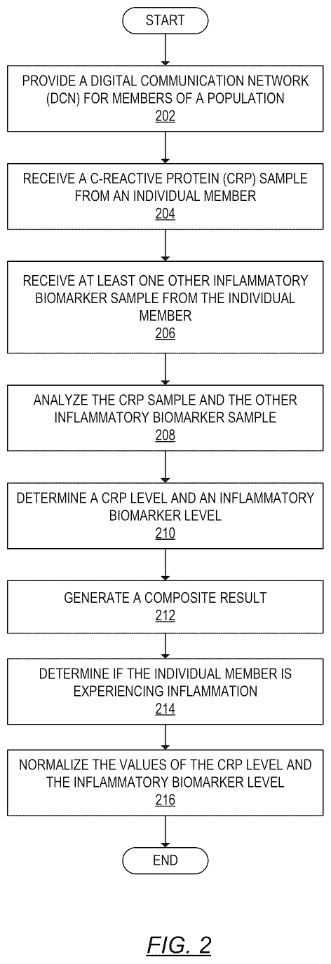

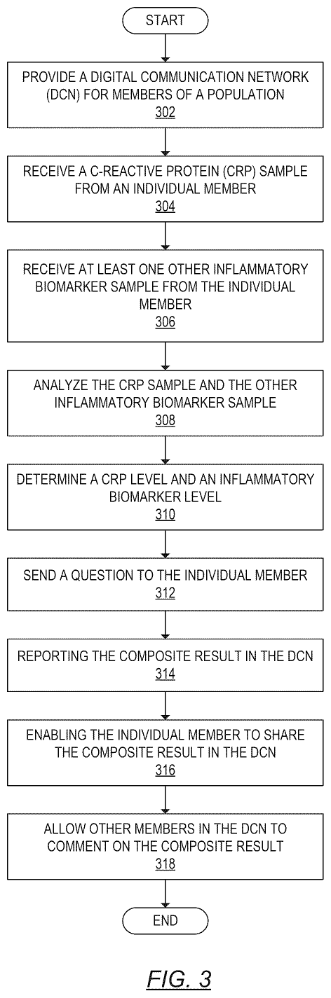

As will be described in more detail in FIGS. 2 and 3, the combination of the CRP level (112) and the inflammation biomarker level (116) can be used measure inflammation in members including instances where measuring CRP levels (112) alone may not be sufficient to detect inflammation.

The data repository (100) also stores a composite result (118). The composite result (118) is a combination of the CRP levels (112), the inflammatory biomarker level (116), or any combination thereof. For example, the composite result (118) can be any of the following: an index (120) created by adding values of the CRP level (112) and the inflammatory biomarker level (116), an index (120) created by providing a highest level of an index created by adding values of the CRP level (112) and the inflammatory biomarker level (116), a spider graph plotting the levels of an index created by adding values of the CRP level (112) and the inflammatory biomarker level (116), an index (120) calculated from the area inside the spider graph plotting the levels of an index created by adding values of the CRP level (112) and the inflammatory biomarker level (116), and an index (120) calculated as the average plus x times the standard deviation of the levels of an index created by adding values of the CRP level (112) and the inflammatory biomarker level (116).

The composite result (118) may inform whether the individual member is experiencing inflammation. For example, if the index (120) of the composite result (118) meets or is greater than a predetermined threshold, then the index (120) may indicate that the individual member is experiencing inflammation. If the index (120) is less than the predetermined threshold, then the index (120) may indicate that the individual member does not have inflammation. The index (120) may also be compared to a set of ranges to determine, for example, a level of inflammation.

By way of background, generalized systemic inflammation is not specific to any disease and, as such, does not often fit into a single category in a healthcare system. As a result, inflammation is not treated in a risk/benefit positive way with drugs or surgery or with any tools that the provider and insurer community provide. Instead, inflammation is a driver and marker of disease progression generally. In some instances, inflammation can be improved (and therefore disease progression can be slowed) with lifestyle, social situation, emotional management with little or no risk.

Although inflammation affects progression, progression is not something the health care system measures. Inflammation and its management impact disease progression and, at some point, disease state. If disease progression can be slowed, the disease state can be avoided, and the population is less sick. State measures have their purpose in decision making but have struggled in terms of slowing the growth of chronic disease. Thus, determining and tracking inflammation via the composite result (118) can be helpful in slowing growth of chronic disease.

The data repository (100) also stores questions (122). The questions (122) are designed to induce a reflection based on the composite result (118). More specifically, the questions (122) may be designed to induce a reflection in the individual member based on lifestyle and environmental factors that caused the composite result (118). For example, the composite result (118) may indicate that the individual member is experiencing inflammation. The inflammation may be due to increased stress and the questions (122) may be designed to induce the individual member to reflect on why the individual member has increased stress.

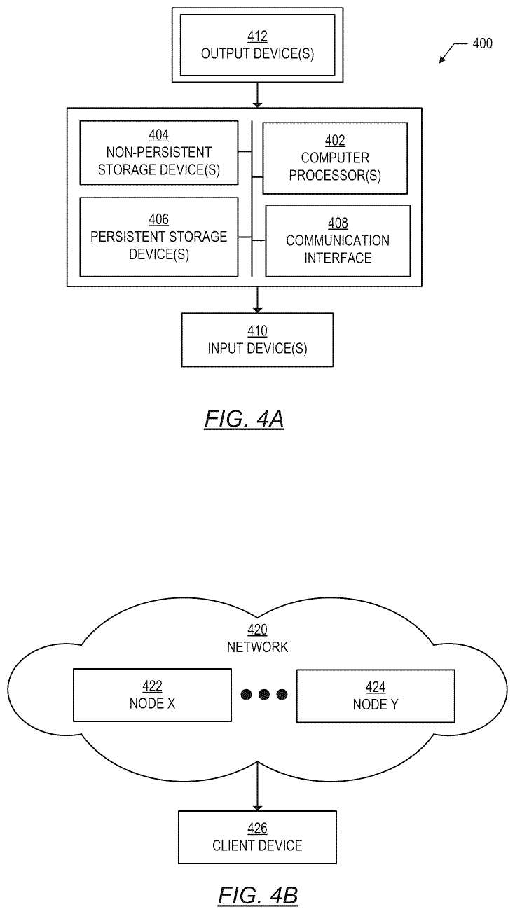

The system shown in FIG. 1 may include other components. For example, the system shown in FIG. 1 also may include a server (130). The server (130) is one or more computer processors, data repositories, communication devices, and supporting hardware and software. The server (130) may be in a distributed computing environment. The server (130) is configured to execute one or more applications, such as a composite result generator (136). An example of a computer system and network that may form the server (130) is described with respect to FIG. 4A and FIG. 4B.

The server (130) also includes a computer processor (132). The computer processor (132) is one or more hardware or virtual processors which may execute computer readable program code that defines one or more applications, such as the composite result generator (136). An example of the computer processor (132) is described with respect to the computer processor(s) (402) of FIG. 4A.

The server (130) also may include a server controller (134). The server controller (134) is software or application specific hardware which, when executed by the computer processor (402), controls and coordinates operation of the software or application specific hardware described herein. Thus, the sever controller (138) may control and coordinate execution of the composite result generator (136).

The server (130) also may include the composite result generator (136). The composite result generator (136) is software or application specific hardware which, when executed by the computer processor (132), receives the CRP level (112) and the inflammatory biomarker level (116) as inputs and outputs the composite result (118).

The server (130) also includes a digital communications network (DCN) (142). The DCN (142) is a network through which members of a population can interact with each other, or with a system supported by the DCN (142). The DCN (142) can, for example, provide sample kits to the members of the population to test for the CRP sample (110) and/or the inflammatory biomarker sample (114). The DCN (142) can also receive the CRP sample (110) and/or the inflammatory biomarker sample (114) from one or more members using the sample kits. The DCN (142) can also provide means for members of the population to communication with each other. Members can, for example, share their composite results (118) and members can also comment on other member's composite results (118) posted.

The members can communicate with each other through the DCN (142). Such communications between members of the population can be one-to-one, one-to-many, one-to-system, system-to-one, or system-to-many. The system may also feature an AI bot or a chatbot that an individual member can share their results (such as, for example, their composite result (118)) with. The members can also converse with the AI bot or chatbot.

The system shown in FIG. 1 also may include one or more user devices (150). The user devices (150) may be considered remote or local. A remote user device is a device operated by a third-party (e.g., an end user of a chatbot) that does not control or operate the system of FIG. 1. Similarly, the organization that controls the other elements of the system of FIG. 1 may not control or operate the remote user device. Thus, a remote user device may not be considered part of the system of FIG. 1.

In contrast, a local user device is a device operated under the control of the organization that controls the other components of the system of FIG. 1. Thus, a local user device may be considered part of the system of FIG. 1.

In any case, the user devices (150) are computing systems (e.g., the computing system (400) shown in FIG. 4A) that communicate with the server (130). The user devices (150) may include a wearable monitor (156) and be configured to send stress indicator data (110) to the server (130). In an alternative embodiment, a separate wearable device may be in communication with the user device (150), such as a smart watch, or blood pressure monitor. The user devices (150) may also include a user input device (152) and/or a display device (154).

In contrast, a local user device is a device operated under the control of the organization that controls the other components of the system of FIG. 1. Thus, a local user device may be considered part of the system of FIG. 1.

While FIG. 1 shows a configuration of components, other configurations may be used without departing from the scope of one or more embodiments. For example, various components may be combined to create a single component. As another example, the functionality performed by a single component may be performed by two or more components.

FIG. 2 is a logic flow diagram that illustrates a method, and a result of execution of computer program instructions, in accordance with various embodiments. The method can be used to slow a progression of an adverse health condition in an individual member of a population. The adverse health condition can be, for example, chronic systemic inflammation, malaise, low energy, a disease, a health risk, social dysfunction, or a prodromal disease. In embodiments where the adverse health condition is a disease, the disease can be, for example obesity, diabetes, rheumatoid arthritis, Crohn's disease, psoriasis, eczema, cardiovascular disease, congestive heart failure, chronic obstructive pulmonary disease, asthma, depression, or anxiety. In embodiments where the adverse health condition is a health risk, the health risk can be, for example falling or becoming infected with an illness.

At Block 202, a step of providing a digital communication network (DCN) for members of a population is provided. The DCN may be the same as or similar to the DCN (142) and provides a network for members of a population to interact with each other or with the DCN.

At Block 204, a step of receiving a C-reactive protein (CRP) sample from an individual member of the population is provided. The CRP sample may be the same as or similar to the CRP sample (110). The CRP sample may be received from an individual member via a user device such as the user device (150). The CRP sample, as previously described, may be obtained by the individual member using an at home test or from a provider at a clinic.

The individual member may submit the CRP sample or may be prompted to submit the CRP sample by, for example, the DCN. In the latter instance, the individual member may be identified by the DCN through review and analysis of the individual member's communications such as posts, messages with other members, etc. The DCN may determine that the individual member can benefit from gathering improved inflammation data through the analysis of the individual member's communications.

At Block 206, a step of receiving at least one other inflammatory biomarker sample from the individual member is provided. The inflammatory biomarker sample may be the same as or similar to the inflammatory biomarker sample (114). The inflammatory biomarker sample may be received from an individual member via the user device. The inflammatory biomarker sample, as previously described, may be obtained by the individual member using an at home test or from a provider at a clinic. The inflammatory biomarker sample may be obtained at the same time as the CRP sample. In other embodiments, the inflammatory biomarker sample may be obtained at a different time than the CRP sample.

At Block 208, a step of analyzing the CRP sample and the other inflammatory biomarker sample is provided. The CRP sample and the inflammatory biomarker sample may be analyzed by, for example, the DCN and/or a composite result generator such as the composite result generator (136).

At Block 210, a step of determining a CRP level and an inflammatory biomarker level is provided. The analysis of Block 208 may be used to determine or measure the CRP level, which may be the same as or similar to the CRP level (112) and the inflammatory biomarker level, which may be the same as or similar to the inflammatory biomarker level (116).

At Block 212, a step of generating a composite result is provided. The composite result may be the same as or similar to the composite result (118). The composite result may be generated by, for example, the composite result generator. As previously described, the composite result can include an index such as the index (120), which can be a combination of the CRP level and the inflammatory biomarker level.

While CRP levels correlate with the inflammatory biomarker levels, testing for CRP levels alone risks missing a change in another marker that might have moved. CRP levels can be combined with the inflammatory biomarker levels, even if most of the time they correlate. Such combination can help catch situations in the small percentage of time when the CRP level and the inflammatory biomarker levels do not correlate. By looking at the combined results of the CRP level and inflammatory biomarker levels, the inflammation data provides a fuller perspective of the subject's health conditions.

At Block 214, a step of determining if the individual member is experiencing inflammation is provided. An index such as the index (120) of the composite result (whether normalized or not) may be compared to a predetermined threshold. When the index meets or exceeds the predetermined threshold, then the individual member is determined to be experiencing inflammation. When the index is below the predetermined threshold, then the individual member is determined to not be experiencing inflammation.

When the individual member is determined to be experiencing inflammation, other steps may be taken. For example, recommendations or interventions for improving their inflammation may be provided to the individual member. The recommendations or interventions may include lifestyle and/or behavior changes that can improve the individual member's inflammation.

At Block 216, a step of normalizing the values of the CRP level and the inflammatory biomarker level is provided. The CRP level and/or the inflammatory biomarker level may be normalized to a value of, for example, 100. In other embodiments, the CRP level and/or the inflammatory biomarker may be normalized to any value such as, for example, the highest normal/acceptable level of the CRP level and/or the inflammatory biomarker level found in humans.

The method of FIG. 2 described above may have more or less steps than shown. Further, steps may be repeated as needed.

FIG. 3 is a logic flow diagram that illustrates a method, and a result of execution of computer program instructions, in accordance with various embodiments. The method can also be used to slow a progression of an adverse health condition in an individual member of a population.

At Block 302, a step of providing a digital communication network (DCN) for members of a population is provided. The step at the Block 302 may be the same as or similar to the Block 202 of FIG. 2 described above.

At Block 304, a step of receiving a C-reactive protein (CRP) sample from an individual member of the population is provided. The step at the Block 304 may be the same as or similar to the Block 204 of FIG. 2 described above.

At Block 306, a step of receiving at least one other inflammatory biomarker sample from the individual member is provided. The step at the Block 306 may be the same as or similar to the Block 206 of FIG. 2 described above.

At Block 308, a step of analyzing the CRP sample and the other inflammatory biomarker sample is provided. The step at the Block 308 may be the same as or similar to the Block 208 of FIG. 2 described above.

At Block 310, a step of determining a CRP level and an inflammatory biomarker level is provided. The step at the Block 310 may be the same as or similar to the Block 210 of FIG. 2 described above.

At Block 312, a step of sending a question to the individual member is provided. The question may be the same as or similar to the question (122). As previously described, the question is designed to induce a reflection in the individual member based on the composite result. More specifically, the question may be designed to induce a reflection in the individual member based on lifestyle and environmental factors that caused the composite result.

At Block 314, a step of reporting the composite result in the DCN is provided. The composite result may automatically be reported in the DCN or reported in the DCN based on input by the individual member. In some embodiments, the composite result may be presented to the individual member to approve prior to reporting the composite result in the DCN.

At Block 316, a step of enabling the individual member to share the composite result in the DCN is provided. Enabling the individual member to share the composite result may include, for example, sharing the results via a conversation bot; allowing the individual member to discuss the results with the conversation bot; and drafting, by the conversation bot, a communication to share with the other members in the DCN. In some embodiments, the conversation bot is designed to have a conversation with the individual member in a way to induce a reflection about the composite result in the individual member.

At Block 318, a step of allowing other members in the DCN to comment on the composite result is provided. Allowing other members to comment on the composite result may include posting the composite result and allowing comments to be posted about the composite result.

The method of FIG. 3 described above may have more or less steps than shown. Further, steps may be repeated as needed.

The methods of FIGS. 2 and 3 can also be used to enable health care in a population to be improved by providing a means for members of said population to gather improved inflammation data through a DCN. For example, communications in the DCN can be analyzed and used to determine to when an individual member might benefit from improved inflammation data. The DCN can then send the message (or messages) to the individual member. In some cases, the process can be iterative, where the individual member's responses to the questions may generate additional questions.

The method of FIGS. 2 and 3 can be used to, for example, measure, track, and manage chronic system inflammation (CSI). For example, specific inflammatory markers may be of interest, e.g., in diagnosing and approving drugs to treat rheumatoid arthritis (RA). The method of FIGS. 2 and 3 can also be used to manage the adverse health condition in the population and/or to reduce the health risk in the population. Management of a condition can include maintaining the condition at the present levels, slowing the progression or reversing the condition (if possible). Using the DCN, many members of the population can be assisted which allows the embodiments to be used to manage the health of the population, the health risks of the population and/or reduce health care expense of the population overall.

CSI can also be used to ascertain the individual member's health conditions which in turn can be used to identify possible actions for the individual member. For example, GPL-1 agonist can be used to jumpstart a fasting regime. Additionally, by empowering members to gain more information about their conditions, the DCN can be used to increase the individual member's awareness of the conditions and more consciously approach their healthcare.

In some embodiments, the system(s) described above may use an AI bot that derives its messages/posts from analysis of communications in the DCN or biometrics provided to the DCN.

Various embodiments also provide a means for the DCN to help an individual member understand their health. For example, the DCN can generate a ‘story’ for the individual member explaining the individual member's situation. The story may be based, at least in part, by communications with the individual member. These communications can be automatically generated from the member's interactions with the DCN, including posts, experiences, biometric results, discussions and the like. The communications can also include conversations with the user through conversational bots. The story may be triggered for generation when the individual member indicates (directly or indirectly) that they have an upcoming interaction with the healthcare system. In this manner, the DCN, through a bot, causes the user to reflect and then assists the user during a meeting with the physician by providing the individual member potential questions to ask the physician.

In various embodiments, the DCN may also use a specialized application, a conversational bot, to communicate with users. The bot may then be used to ask the users questions in which to base their story. One-to-one communications with the conversational bot can be triggered and informed by one-to-many communications in the DCN, and conversations with the conversational bot can (a) refer individual members to the relevant communications in the DCN, (b) suggest posts to the DCN, and (c) make reference to ideas contained in posts. The two ways to interact can behave in a synergistic way and allow the conversational bot to include a “human in the loop” in a surprisingly scalable way.

One or more embodiments may be implemented on a computing system specifically designed to achieve an improved technological result. When implemented in a computing system, the features and elements of the disclosure provide a significant technological advancement over computing systems that do not implement the features and elements of the disclosure. Any combination of mobile, desktop, server, router, switch, embedded device, or other types of hardware may be improved by including the features and elements described in the disclosure.

For example, as shown in FIG. 4A, the computing system (400) may include one or more computer processor(s) (402), non-persistent storage device(s) (404), persistent storage device(s) (406), a communication interface (408) (e.g., Bluetooth interface, infrared interface, network interface, optical interface, etc.), and numerous other elements and functionalities that implement the features and elements of the disclosure. The computer processor(s) (402) may be an integrated circuit for processing instructions. The computer processor(s) (402) may be one or more cores, or micro-cores, of a processor. The computer processor(s) (402) includes one or more processors. The computer processor(s) (402) may include a central processing unit (CPU), a graphics processing unit (GPU), a tensor processing unit (TPU), combinations thereof, etc.

The input device(s) (410) may include a touchscreen, keyboard, mouse, microphone, touchpad, electronic pen, or any other type of input device. The input device(s) (410) may receive inputs from a user that are responsive to data and messages presented by the output device(s) (412). The inputs may include text input, audio input, video input, etc., which may be processed and transmitted by the computing system (400) in accordance with one or more embodiments. The communication interface (408) may include an integrated circuit for connecting the computing system (400) to a network (not shown) (e.g., a local area network (LAN), a wide area network (WAN) such as the Internet, mobile network, or any other type of network) or to another device, such as another computing device, and combinations thereof.

Further, the output device(s) (412) may include a display device, a printer, external storage, or any other output device. One or more of the output device(s) (412) may be the same or different from the input device(s) (410). The input device(s) (410) and output device(s) (412) may be locally or remotely connected to the computer processor(s) (402). Many different types of computing systems exist, and the aforementioned input device(s) (410) and output device(s) (412) may take other forms. The output device(s) (412) may display data and messages that are transmitted and received by the computing system (400). The data and messages may include text, audio, video, etc., and include the data and messages described above in the other figures of the disclosure.

Software instructions in the form of computer readable program code to perform embodiments may be stored, in whole or in part, temporarily or permanently, on a non-transitory computer readable medium such as a solid-state drive (SSD), compact disk (CD), digital video disk (DVD), storage device, a diskette, a tape, flash memory, physical memory, or any other computer readable storage medium. Specifically, the software instructions may correspond to computer readable program code that, when executed by the computer processor(s) (402), is configured to perform one or more embodiments, which may include transmitting, receiving, presenting, and displaying data and messages described in the other figures of the disclosure.

The computing system (400) in FIG. 4A may be connected to, or be a part of, a network. For example, as shown in FIG. 4B, the network (420) may include multiple nodes (e.g., node X (422) and node Y (424), as well as extant intervening nodes between node X (422) and node Y (424)). Each node may correspond to a computing system, such as the computing system shown in FIG. 4A, or a group of nodes combined may correspond to the computing system shown in FIG. 4A. By way of an example, embodiments may be implemented on a node of a distributed system that is connected to other nodes. By way of another example, embodiments may be implemented on a distributed computing system having multiple nodes, where each portion may be located on a different node within the distributed computing system. Further, one or more elements of the aforementioned computing system (400) may be located at a remote location and connected to the other elements over a network.

The nodes (e.g., node X (422) and node Y (424)) in the network (420) may be configured to provide services for a client device (426). The services may include receiving requests and transmitting responses to the client device (426). For example, the nodes may be part of a cloud computing system. The client device (426) may be a computing system, such as the computing system shown in FIG. 4A. Further, the client device (426) may include or perform all or a portion of one or more embodiments.

The computing system of FIG. 4A may include functionality to present data (including raw data, processed data, and combinations thereof) such as results of comparisons and other processing. For example, presenting data may be accomplished through various presenting methods. Specifically, data may be presented by being displayed in a user interface, transmitted to a different computing system, and stored. The user interface may include a graphical user interface (GUI) that displays information on a display device. The GUI may include various GUI widgets that organize what data is shown, as well as how data is presented to a user. Furthermore, the GUI may present data directly to the user, e.g., data presented as actual data values through text, or rendered by the computing device into a visual representation of the data, such as through visualizing a data model.

Various operations described are purely exemplary and imply no particular order. Further, the operations can be used in any sequence when appropriate and can be partially used. With the above embodiments in mind, it should be understood that additional embodiments can employ various computer-implemented operations involving data transferred or stored in computer systems. These operations are those requiring physical manipulation of physical quantities. Usually, though not necessarily, these quantities take the form of electrical, magnetic, or optical signals capable of being stored, transferred, combined, compared, and otherwise manipulated.

Any of the operations described that form part of the presently disclosed embodiments may be useful machine operations. Various embodiments also relate to a device or an apparatus for performing these operations. The apparatus can be specially constructed for the required purpose, or the apparatus can be a general-purpose computer selectively activated or configured by a computer program stored in the computer. In particular, various general-purpose machines employing one or more processors coupled to one or more computer readable medium, described below, can be used with computer programs written in accordance with the teachings herein, or it may be more convenient to construct a more specialized apparatus to perform the required operations.

The procedures, processes, and/or modules described herein may be implemented in hardware, software, embodied as a computer-readable medium having program instructions, firmware, or a combination thereof. For example, the functions described herein may be performed by a processor executing program instructions out of a memory or other storage device.

The foregoing description has been directed to particular embodiments. However, other variations and modifications may be made to the described embodiments, with the attainment of some or all of their advantages. Modifications to the above-described systems and methods may be made without departing from the concepts disclosed herein. Accordingly, the invention should not be viewed as limited by the disclosed embodiments. Furthermore, various features of the described embodiments may be used without the corresponding use of other features. Thus, this description should be read as merely illustrative of various principles, and not in limitation of the invention.

What is claimed is:

1. A method for managing an adverse health condition, the method comprising:
providing a digital communication network (DCN) for a plurality of members of a population to interact with each other and the DCN;
receiving a C-reactive protein (CRP) sample from an individual member of a plurality of members of a population;
receiving at least one other inflammatory biomarker sample from the individual member;
analyzing the CRP sample and the at least one other inflammatory biomarker;
determining a CRP level from the CRP sample and an inflammatory biomarker level from the at least one other inflammatory biomarker sample;
generating a composite result based on the CRP level and the inflammatory biomarker level; and
providing the composite result to the individual member via the DCN,
wherein the composite result correlates to whether the individual member is experiencing inflammation.

2. The method of claim 1, wherein the composite result comprises at least one of:
an index created by adding values of the CRP level and the inflammatory biomarker level,
an index created by providing a highest level of an index created by adding values of the CRP level and the inflammatory biomarker level,
a spider graph plotting the levels of an index created by adding values of the CRP level and the inflammatory biomarker level,
an index calculated from the area inside the spider graph plotting the levels of an index created by adding values of the CRP level and the inflammatory biomarker level, and
an index calculated as the average plus x times the standard deviation of the levels of an index created by adding values of the CRP level and the inflammatory biomarker level.

3. The method of claim 2, wherein the CRP level and the inflammatory biomarkers level are normalized.

4. The method in claim 3, further comprising normalizing the values of the CRP level and the inflammatory biomarker level.

5. The method in claim 1, further comprising comparing an index of the composite result with a predetermined threshold and determining that the individual member is experiencing inflammation when the index meets the predetermined threshold.

6. The method of claim 1, wherein the at least one other inflammatory biomarker is at least one of: TNF-alpha, IL-1 Beta, IL-6, IL-8, IL-10, IL-17, IL-17a, IL-12, INF, IL-23, Erythrocyte Sedimentation Rate, Plasma Viscosity, Fibringen, Ferritin, Procalcitonin, Psychometric Instruments of depression, anxiety, stress, and cognitive load, and Fasting and postprandial Insulin.

7. The method of claim 1, wherein the adverse health condition comprises at least one of chronic systemic inflammation, malaise, low energy, a disease, a health risk, social dysfunction, or a prodromal disease.

8. The method of claim 7, wherein the disease comprises at least one of obesity, diabetes, rheumatoid arthritis, Crohn's disease, psoriasis, eczema, cardiovascular disease, congestive heart failure, chronic obstructive pulmonary disease, asthma, depression, or anxiety.

9. The method of claim 7, wherein the health risk is at least one of falling or becoming infected with an illness.

10. The method of claim 1, wherein the method is used to measure chronic systemic inflammation.

11. The method of claim 1, wherein the method is used to manage the adverse health condition in the population.

12. The method of claim 1, wherein the method is used to reduce the health risk in the population.

13. The method of claim 1, further comprising sending a question to the individual member from the DCN, wherein the question is designed to induce a reflection based on the composite result.

14. The method of claim 13, wherein the question is further designed to induce the reflection based on lifestyle and environmental factors that caused the composite result.

15. The method of claim 1, further comprising reporting the composite result in the DCN.

16. The method of claim 15, further comprising enabling the individual member to share the composite result with other members in the DCN.

17. The method of claim 15, further comprising allowing the other members in the DCN to comment upon the composite result.

18. The method of claim 16, wherein enabling the individual member to share the composite result comprises at least one of:
sharing the results via a conversation bot;
allowing the member to discuss the results with the conversation bot; and
drafting, by the conversation bot, a communication to share with the other members in the DCN.

19. The method of claim 18, wherein the conversation bot is designed to have a conversation configured to induce a reflection about the composite result in the member.

20. A system comprising:
a computer processor;
a data repository in communication with the computer processor and storing:
a C-reactive protein (CRP) sample,
a CRP level,
an inflammatory biomarker sample,
an inflammatory biomarker level, and
a composite result,
a composite result generator configured to receive the CRP level and the inflammatory biomarker level as input and to output the composite result;
a digital communications network which, when executed by the computer processor, provides a network for members of a population to interact with each other; and
a server controller which, when executed by the computer processor:
provide a digital communication network (DCN) for a plurality of members of a population to interact with each other and the DCN;
receive the CRP sample from an individual member of a plurality of members of a population;
receive the inflammatory biomarker sample from the individual member;
analyze the CRP sample and the at least one other inflammatory biomarker;
determine the CRP level from the CRP sample and the inflammatory biomarker level from the inflammatory biomarker sample;
generate the composite result based on the CRP level and the inflammatory biomarker level; and
provide the composite result to the individual member via the DCN.
